(19) United States
(12) Patent Application Publication
(10) Pub. No.: US 2008/0210230 A1
(43) Pub. Date: Sep. 4, 2008

(54) Inhalation Devices

(75) Inventors: Richard David Lintern, Cambridgeshire (GB); Paul Kenneth Rand, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: Glaxo Group Limited, Greenford Middlesex (GB)

(21) Appl. No.: 11/908,797

(22) PCT Filed: Mar. 16, 2006
(86) PCT No.: PCT/GB2006/000965

§ 371 Date: Sep. 17, 2007

(30) Foreign Application Priority Data
Mar. 17, 2005 (GB) 0505538.9

(51) Int. Cl. A61M 11/00

(52) U.S. Cl. 128/200.23

(57) ABSTRACT

An inhaler for delivering medicament by inhalation, comprising: a canister which comprises a body which defines a chamber containing medicament and a valve stem which extends from the body and from which medicament is in use delivered on actuation of the canister; and an actuator comprising a main body comprising a housing receiving the canister and actuating mechanism for actuating the canister, wherein the actuating mechanism comprises a loading member which engages or is comprised in the canister, and at least one actuating member which is depressible by a user to drive the loading member in an actuating direction from a first, rest position to a second, actuated position in which the canister is actuated to deliver medicament, wherein the at least one actuating member is a flexible element which has a first, flexed configuration when the loading member is in the first, rest position and, on depression, is extended in the actuating direction, such as to drive the loading member in the actuating direction from the first, rest position to the second, actuated position.

### FIELD OF THE INVENTION

The present invention relates to an actuator for an inhaler for administering medicament by inhalation and to an inhaler including the same. The invention is particularly, but not exclusively, concerned with an actuator for a pressurised metered dose inhaler (pMDI).

### BACKGROUND OF THE INVENTION

pMDIs are well known in the art of inhalation devices. It is therefore not necessary to describe the construction and operation of a pMDI other than in bare essentials.

A pMDI comprises a canister and an actuator housing. The housing is generally tubular, although this is not essential, and generally formed of a plastics material, for instance by moulding. The canister comprises an open-ended canister, typically made from a metal such as aluminium. The open end of the canister is sealingly capped by a metering valve assembly. The valve assembly typically includes a hollow dispensing member or valve stem which projects from the outlet or business end of the canister. The dispensing member is mounted for sliding movement relative to the canister between an extended position, to which the dispensing member is biased by a biasing mechanism in the valve assembly, typically a return spring, and a depressed position.

In use, the sealed canister contains a pressurised medicinal aerosol formulation. The formulation comprises the medicament and a fluid propellant, and optionally one or more excipients and/or adjuvants. The medicament is typically in solution or suspension in the formulation. The propellant is typically a CFC-free propellant, suitably a liquid propellant, and may for example be HFA-134a or HFA-227.

Movement of the dispensing member from the extended position to the depressed position results in a metered dose of the aerosol formulation being dispensed from the canister through the dispensing member. Typically, the metering valve assembly is provided with a metering chamber of defined volume. In the extended position of the dispensing member, the content of the canister is placed in fluid communication with the metering chamber through the dispensing member so that the metering chamber is filled with the aerosol formulation. When the dispensing member is depressed, the metering chamber is isolated from the canister inner volume and placed in fluid communication with the external environment through the dispensing member. Thus, the defined volume of the aerosol formulation in the metering chamber is discharged to the external environment via the dispensing member.

Such metering valve assemblies are well known in the art and can be obtained from inter alia Bespak Plc (King's Lynn, Norfolk, United Kingdom) and Valois S.A.S. (Le Neubourg, France).

The housing typically comprises an internal passageway having an open end. The canister is slidable into the internal passageway through the open end with the canister being inserted valve assembly first into the internal passageway. A stem block, which receives the dispensing member of the canister when the canister is received in the housing in a “rest position”, has a passageway with an inlet end for receiving the dispensing member and an outlet end, which faces a dispensing outlet of the housing, typically a mouthpiece or a nasal nozzle. The stem block holds the dispensing member stationary whereby depression of the canister to its rest position further into the housing to an “actuated position” causes the dispensing member to be displaced from the extended position to the depressed position relative to the canister. A metered dose of the aerosol formulation will thereby be dispensed out of the dispensing outlet of the housing via the internal passageway of the stem block.

In use, a patient in need of a metered dose of the medicinal aerosol formulation concurrently inhales on the dispensing outlet and depresses the canister from the rest position to the actuated position. The inspiratory airflow produced by the patient entrains the metered dose of the medicinal aerosol formulation into the patient's respiratory tract. So, a PMDI of the type described above is a breath-coordinated inhaler.

Inhalers are commonly provided with a dust cap that covers the dispensing outlet when the inhaler is not in use. The dust cap, when applied, prevents foreign material from entering the housing. This prevents the user from inhaling dust or lint, for example, that might otherwise accumulate in the housing. This is of particular importance where the user suffers from asthma or other respiratory conditions, in which the inhalation of foreign material may cause severe irritation.

Developments to pMDIs have included the provision of actuation indicators or dose counters therefor. Such a dose counter is described in PCT Patent Application Nos. WO-A-9856444 and WO-A-2004/001664 to Glaxo Group Limited. The dose counter is fixably secured on the valve assembly end of the canister and includes a display which denotes the number of metered doses of the medicament formulation dispensed from, or remaining in, the canister. The display of the dose counter is visible to the patient through a window provided in the housing. The display may be presented by a plurality of indicator wheels rotatably mounted on a common axle, each wheel having numerals displayed in series around the circumference.

Many actuators for inhalers have been developed with a view to facilitating the delivery of medicament, examples of which are disclosed in U.S. Pat. No. 3,272,391, U.S. Pat. No. 3,272,392, U.S. Pat. No. 4,678,106, U.S. Pat. No. 5,899,365, U.S. Pat. No. 6,237,812 and WO-A-99/49917.

It is an aim of the present invention to provide an improved actuator for an inhaler for administering medicament by inhalation and an inhaler including the same.

### SUMMARY OF THE INVENTION

In one aspect the present invention provides an inhaler for delivering medicament by inhalation according to claim 1 hereof.

Preferably, the main body includes at least one engagement element which engages the at least one actuating member.

In one embodiment the housing of the main body includes the at least one engagement element.

In one embodiment the at least one engagement element on the main body engages an end of the at least one actuating member.

Preferably, the loading member includes at least one engagement element which engages the at least one actuating member.

In one embodiment the at least one engagement element on the loading member engages an end of the at least one actuating member.

In one embodiment the body of the canister includes a base and a head, and the loading member is attached to the head of the body of the canister.

Preferably, the loading member comprises a sleeve in which the head of the body of the canister is fixed and a body section which includes the at least one engagement element.

In another embodiment the body of the canister includes a base and a head, and the loading member is located over the base of the body of the canister.

Preferably, the loading member comprises a sleeve which fits about an outer peripheral surface of the body of the canister and includes the at least one engagement element, and an end section at one end of the sleeve which engages the base of the body of the canister.

In one embodiment the at least one engagement element is disposed at the other end of the sleeve of the loading member.

Preferably, the sleeve of the loading member extends substantially to the head of the body of the canister.

Preferably, the at least one actuating member is an elongate element.

In one embodiment the at least one actuating member is a resilient element which adopts the flexed configuration when not depressed.

In one embodiment the housing includes at least one lateral opening in which the at least one actuating member is disposed for depression by the user.

In another embodiment the main body further comprises at least one grip member which is movably disposed relative to the housing, such as, on depression, to engage the at least one actuating member in actuating the actuating mechanism.

Preferably, the at least one grip member is pivotally coupled to the housing.

Preferably, the actuating mechanism comprises first and second actuating members.

More preferably, the actuating members are disposed to opposite lateral sides of the canister.

Preferably, the main body includes a nozzle block which receives the valve stem of the canister.

Preferably, the housing includes an outlet member through which the user in use inhales.

More preferably, the outlet member is a mouthpiece.

In another aspect of the present invention there is provided the actuator of the inhaler of the invention.

Other aspects and features of the invention are set forth in the appended claims and the exemplary embodiments which will now be described with reference to the accompanying Figures of drawings.

### BRIEF DESCRIPTION OF THE DRAWINGS

FIG. 1 illustrates a perspective view of an inhaler in accordance with a first embodiment of the present invention;

FIG. 2 illustrates an exploded perspective view of the inhaler of FIG. 1;

FIG. 3 illustrates a part-sectional view of the inhaler of FIG. 1, where illustrated in the inoperative, rest configuration;

FIG. 4 illustrates a part-sectional view of the inhaler of FIG. 1, where illustrated in the actuated configuration;

FIG. 5 illustrates a perspective view of an inhaler in accordance with a second embodiment of the present invention;

FIG. 6 illustrates a part-sectional view of the inhaler of FIG. 5, where illustrated in the inoperative, rest configuration; and

FIG. 7 illustrates a part-sectional view of the inhaler of FIG. 5, where illustrated in the actuated configuration.

### DETAILED DESCRIPTION OF EXEMPLARY EMBODIMENTS

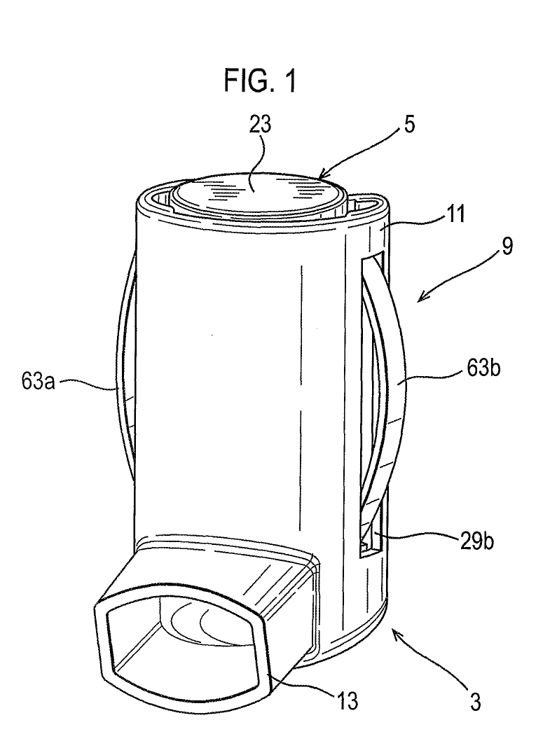

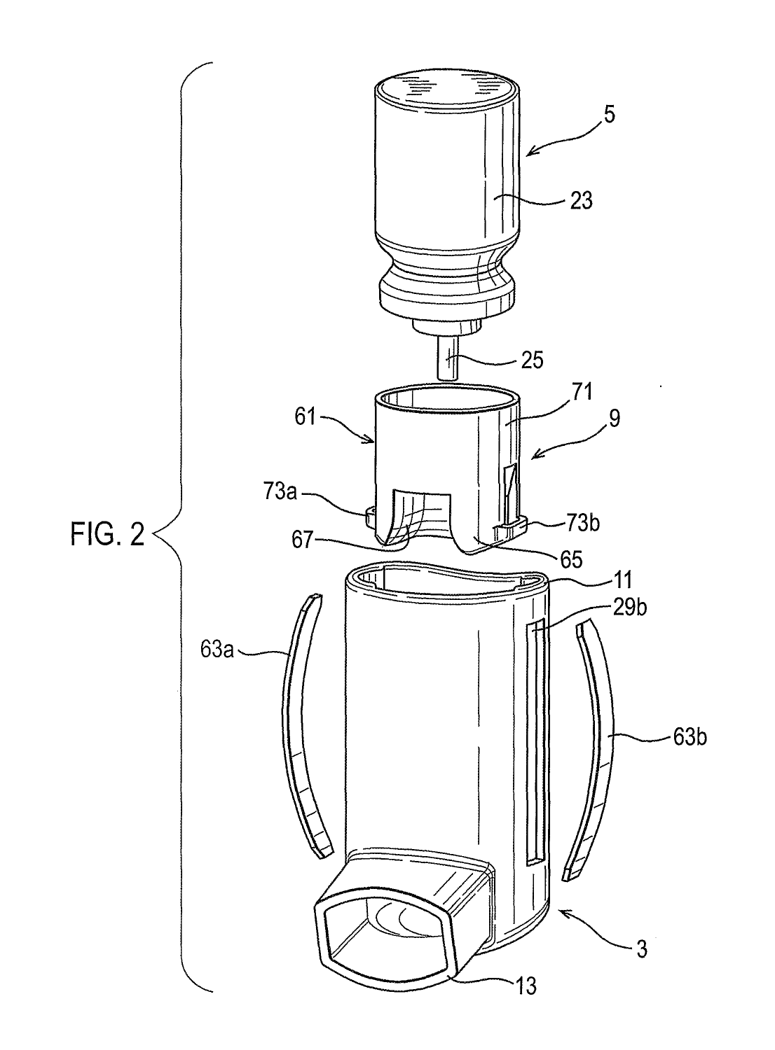

FIGS. 1 to 4 illustrate a hand-held, hand-operable inhaler of the pMDI type in accordance with a first embodiment of the present invention.

The inhaler comprises an actuator which comprises a main body 3, an aerosol canister 5 which is fitted in the main body 3 and contains medicament to be delivered on actuation of the inhaler, and an actuating mechanism 9 which is operable by a user to actuate the inhaler.

The main body 3 comprises a housing 11 in which the canister 5 is in use fitted, and a mouthpiece 13, in this embodiment a tubular element, which is in fluid communication with one, the lower, end of the housing 11 and in use is gripped in the lips of the user. The mouthpiece 13 could instead be configured as a nasal nozzle. The main body 3 in this embodiment is of a plastics material, for instance formed by moulding.

The canister 5 in this embodiment is of standard type, as outlined supra, and comprises a body 23 which includes a base and a head and defines a chamber containing a medicament in a CFC-free propellant under pressure, for example an HFA propellant, a valve stem 25 which extends from the head of the body 23 and an internal metering valve (not illustrated) which is normally biased by an internal valve spring (not illustrated) to a closed position and opened to deliver a metered dose of medicament from the canister 5 when the valve stem 25 is depressed into the canister body 23.

The housing 11 includes first and second lateral apertures 29a, b, in this embodiment elongate apertures which extend between the ends of the housing 11 and are disposed in opposed relation to lateral sides of the mouthpiece 13 and receive actuating members 63a, b of the actuating mechanism 9, as will be described in more detail hereinbelow.

The housing 11 further includes first and second engagement elements 31a, b which are disposed at the respective upper ends of the lateral apertures 29a, b. In this embodiment the engagement elements 31a, b each comprise a recess, here in the form of a slot, in which the upper end of a respective one of the actuating members 63a, b is located, as will be described in more detail hereinbelow.

The main body 3 further comprises a nozzle block 33, in this embodiment disposed to a base surface of the housing 11, for receiving the valve stem 25 of the canister 5.

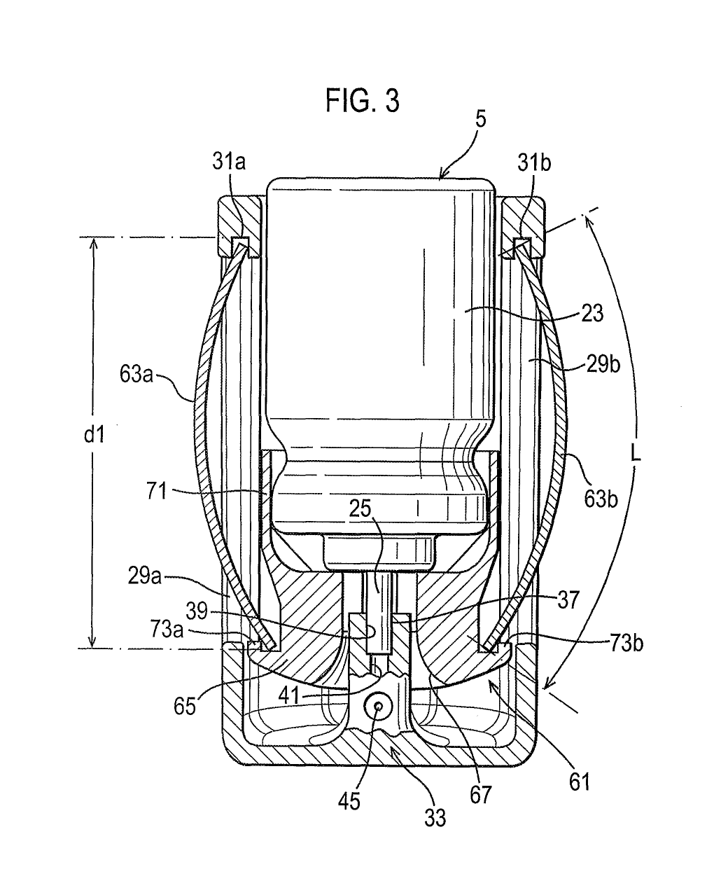

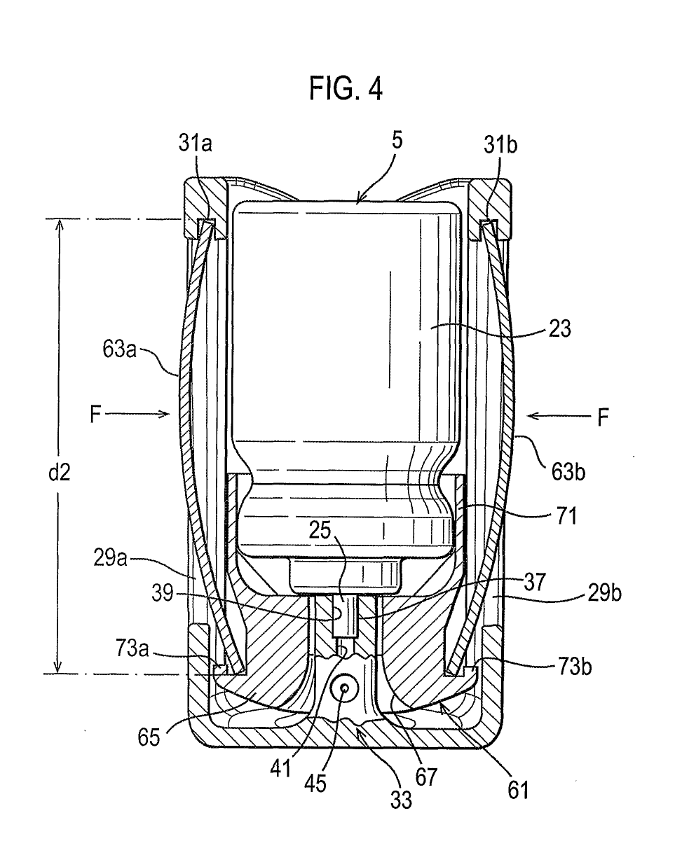

Referring particularly to FIGS. 3 and 4, the nozzle block 33 includes a tubular bore 37 for receiving the valve stem 25 of the canister 5, which in this embodiment is co-axial with the longitudinal axis of the housing 11. The tubular bore 37 is open at one, the upper, end thereof and includes an upper section 39 which has an internal dimension which is substantially the same as the outer dimension of the valve stem 25 and a lower section 41 which has a smaller dimension, which sections 39, 41 together define an annular seat for the distal end of the valve stem 25. The tubular bore 37 further includes a laterally-directed spray orifice 45 in the lower section 41 thereof which is configured to direct a spray of the medicament formulation dispensed from the valve stem into and through the mouthpiece 13.

The actuating mechanism 9 comprises a loading member 61 which is attached to the head of the body 23 of the canister 5, and first and second actuating members 63a, b which are disposed at the lateral apertures 29a, b in the housing 11 and operable to provide for the loading member 61, and hence the body 23 of the canister 5, to be driven downwardly on depression of the actuating members 63a, b. The components of the actuating mechanism may be of a plastics material.

The loading member 61 may be a dose counter, for example of the type described in WO-A-9856444 and WO-A-2004/001664 supra, and may be attached to the canister 5 as further detailed in these publications which are hereby incorporated herein by reference in their entirety.

In this embodiment the loading member 61 is slideably disposed over the nozzle block 33 between a first, rest or inoperative position, as illustrated in FIG. 3, and a second, actuated position in which the canister 5 is actuated, as illustrated in FIG. 4, and comprises a body section 65 which includes a through bore 67 in which the nozzle block 33 is slideably disposed, and a sleeve 71, here tubular, in which the head of the body 23 of the canister 5 is fixed, such that the body 23 of the canister 5 is moved together with the loading member 61.

In this embodiment the body section 65 includes first and second engagement elements 73a, b which are disposed to opposite sides of the body section 65 and positioned radially such as to be adjacent the lateral apertures 29a, b in the housing 11. The engagement elements 73a, b are positioned axially on the body section 65 such that the distance in the axial direction therefrom to the engagement elements 31a, b on the housing 11 is a first distance d₁ when the loading member 61 is in the rest position and a second distance d₂, which is greater than the first distance d₁, when the loading member 61 is in the actuated position.

The loading member 61 is in its rest position when the valve stem 25 is inserted in the nozzle block 33 and the return spring (not shown) in the metering valve assembly has biased or returned the canister-loading member unit 5, 61 to the position in the main body 3 shown in FIG. 3.

In this embodiment the engagement elements 73a, b on the loading section 65 each comprise a recess, here in the form of a slot, in which the lower end of a respective one of the actuating members 63a, b is located, as will be described in more detail hereinbelow.

The actuating members 63a, b each comprise a flexible, elongate element, which has a length l which is greater both than the first distance d₁ and the second distance d₂ between the engagement elements 31a, b on the housing 11 and the engagement elements 73a, b on the loading member 61, the respective ends of which engage respective ones of the engagement elements 31a, b on the housing 11 and the engagement elements 73a, b on the loading member 61. With this configuration, as illustrated in FIG. 3, the actuating members 63a, b are each flexed outwardly when the actuating mechanism 9 is in the rest configuration, thus presenting the actuating members 63a, b for depression by the user (e.g. with opposing digits of a user's hand), and, when depressed, the actuating members 63a, b are straightened, such as to extend a greater distance in the axial direction, and, when this extension reaches the second distance d₂ between the engagement elements 31a, b on the housing 11 and the engagement elements 73a, b on the loading member 61, the canister 5 is actuated.

In this embodiment the actuating members 63a, b comprise resilient elements, typically formed or a metal or plastic, which adopt a flexed configuration, as illustrated in FIG. 3, when the actuating mechanism 9 is in the rest configuration.

Operation of the actuator will now be described hereinbelow.

The user first takes the actuator, as illustrated in FIG. 1, in one hand.

The user then takes the mouthpiece 13 in his/her lips, and, in co-ordination with an inhalation breath, actuates the inhaler by depressing the actuating members 63a, b of the actuating mechanism 9.

As illustrated in FIG. 4, depression of the actuating members 63a, b causes the actuating members 63a, b to be straightened and thereby extended in the axial direction.

This straightening and extension in the axial direction of the actuating members 63a, b causes the loading member 61, and hence the body 23 of the canister 5 which is fixed thereto, to be driven downwardly in relation to the stationary valve stem 25 of the canister 5.

When this extension reaches the second distance d₂ between the engagement elements 31a, b on the housing 11 and the engagement elements 73a, b on the loading member 61, the canister 5 is actuated to deliver a spray of the medicament formulation into and through the mouthpiece 13.

Following actuation, the inhaler is removed from the mouth, and the actuating members 63a, b are released. On releasing the actuating members 63a, b, the actuating mechanism 9 is returned by the valve return spring to the rest configuration, as illustrated in FIGS. 1 and 3, ready for subsequent actuation.

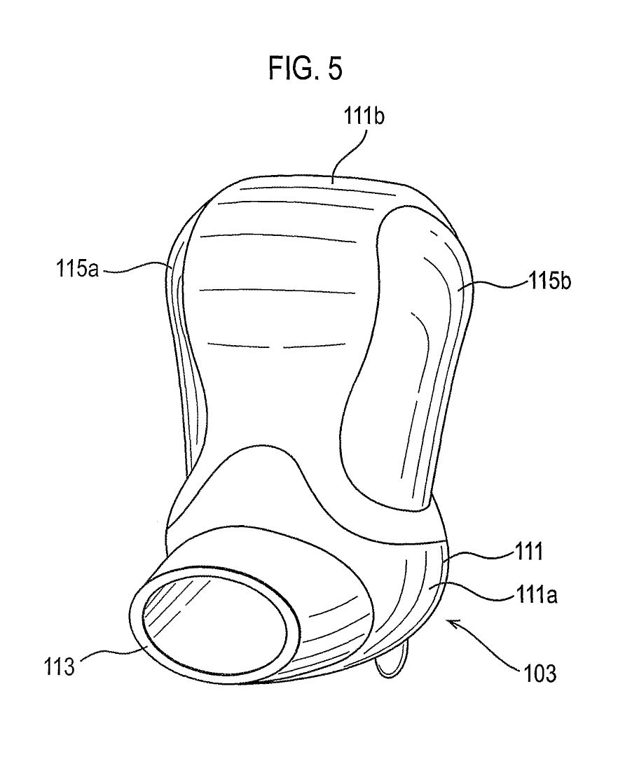

FIGS. 5 to 7 illustrate a hand-held, hand-operable inhaler of the pMDI type in accordance with a second embodiment of the present invention. Those parts of the second embodiment corresponding to parts in the first embodiment are assigned like reference numerals.

The inhaler comprises an actuator which comprises a main body 103, an aerosol canister 105 which is fitted in the main body 103 and contains medicament to be delivered on actuation of the inhaler, and an actuating mechanism 109 which is operable by a user to actuate the inhaler.

The main body 103 comprises a housing 111 in which the canister 105 is in use fitted, a mouthpiece 113, in this embodiment a tubular element, which is in fluid communication with one, the lower, end of the housing 111 and in use is gripped in the lips of the user, and first and second grip members 115a, b which are movably disposed to the housing 111 such as to be depressed by the user in actuating the actuating mechanism 109, as will be described in more detail hereinbelow.

The canister 105 corresponds to that in FIGS. 1 to 4 and comprises a body 123 which includes a base and a head and defines a chamber containing a medicament in a propellant under pressure (e.g. HFA), a valve stem 125 which extends from the head of the body 123 and an internal metering valve (not illustrated) which is normally biased to a closed position and opened to deliver a metered dose of medicament from the canister 105 when the valve stem 125 is depressed.

In this embodiment the housing 111 comprises first and second housing parts 111a, b which are attached together, here by clips.

The first housing part 111a is in fluid communication with the mouthpiece 113, and includes a nozzle block 133, in this embodiment disposed to a base surface of the first housing part 111a, for receiving the valve stem 125 of the canister 105. In this embodiment the first housing part 111a is formed, here by moulding, as a single, integral unit.

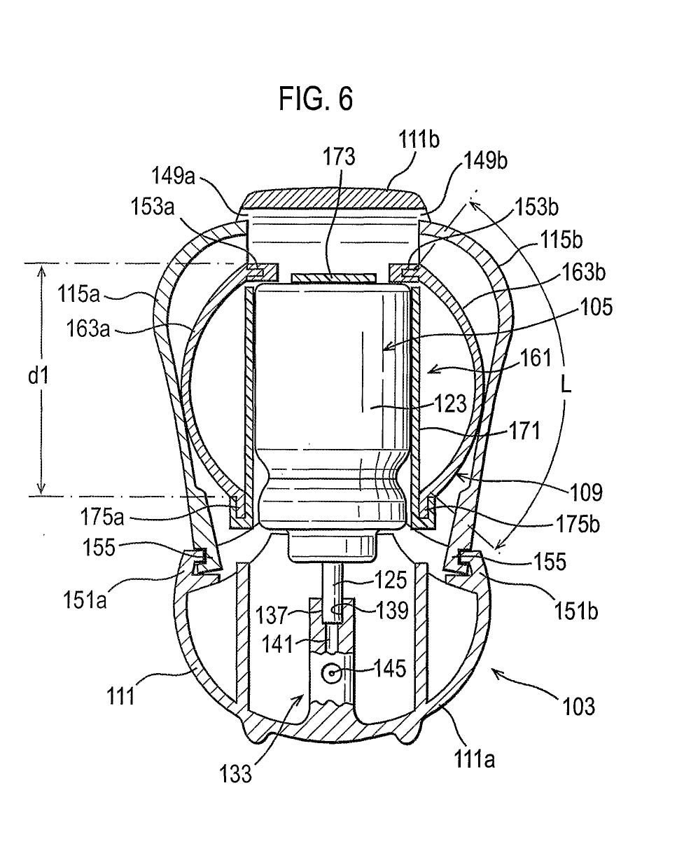

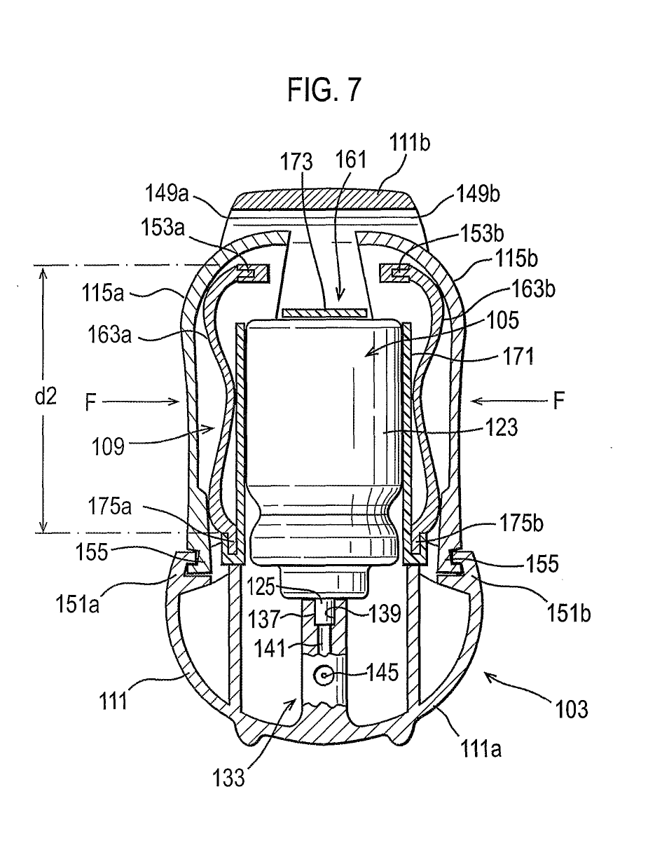

Referring particularly to FIGS. 6 and 7, the nozzle block 133 includes a tubular bore 137 for receiving the valve stem 125 of the canister 105, which in this embodiment is co-axial with the longitudinal axis of the housing 111. The tubular bore 137 is open at one, the upper, end thereof and includes an upper section 139 which has an internal dimension which is substantially the same as the outer dimension of the valve stem 125 and a lower section 141 which has a smaller dimension, which sections 139, 141 together define an annular seat for the distal end of the valve stem 125. The tubular bore 137 further includes a laterally-directed spray orifice 145 in the lower section 141 thereof which is configured to direct a spray into and through the mouthpiece 113.

The second housing part 111b includes first and second lateral apertures 149a, b, in this embodiment elongate apertures which extend between the ends of the second housing part 111b, which are disposed in opposed relation to lateral sides of the mouthpiece 113 and receive the grip members 115a, b.

The second housing part 111b further includes first and second pivot elements 151a, b which are disposed at the respective lower ends of the lateral apertures 149a, b. In this embodiment the pivot elements 151a, b each comprise a recess, here in the form of a slot, in which the lower end of a respective one of the grip members 115a, b is located, as will be described in more detail hereinbelow.

The upper housing part 111b further includes first and second engagement elements 153a, b which are disposed at the respective upper ends of the lateral apertures 149a, b. In this embodiment the engagement elements 153a, b each comprise a recess, here in the form of a slot, in which the upper end of an actuating member 163a, b of the actuating mechanism 109 is located, as will be described in more detail hereinbelow.

In this embodiment the grip members 115a, b each comprise an elongate element in the form of a lever, which includes a pivot element 155 at the lower end thereof, which is engaged in a respective one of the pivot elements 151a, b on the housing 111, such as to provide for the pivoting of the respective grip member 115a, b relative to the housing 111.

The actuating mechanism 109 comprises a loading member 161, in this embodiment of a plastics material, which is fitted over the base of the body 123 of the canister 105, and first and second actuating members 163a, b which are disposed at the lateral apertures 149a, b in the housing 111 such as to be engaged by the respective ones of the grip members 115a, b, and operable to provide for the loading member 161, and hence the body 123 of the canister 105, to be driven downwardly on depression of the actuating members 163a, b.

In this embodiment the loading member 161 is slideably disposed relative to the nozzle block 133 between a first, rest or inoperative position to which it is biased by the return spring (not shown) of the metering valve assembly of the canister 105, as illustrated in FIG. 6, and a second, actuated position in which the canister 105 is actuated, as illustrated in FIG. 7, and comprises a sleeve 171, here tubular, which is a close fit with the outer peripheral wall of the body 123 of the canister 105 and extends over substantially the entire length of the body 123 of the canister 105 from the base to the head of the body 123 of the canister 105, an end section 173 at one, the upper, end of the sleeve 171, here which spans the sleeve 171, which engages the base of the body 123 of the canister 105, and first and second engagement elements 175a, b which are disposed at the other, lower end of the sleeve 171 to opposite sides of the sleeve 171 and positioned radially such as to be adjacent the lateral apertures 149a, b in the housing 111. The engagement elements 175a, b are positioned axially on the sleeve 171 such that the distance in the axial direction therefrom to the engagement elements 153a, b on the housing 111 is a first distance d₁ when the loading member 161 is in the rest position and a second distance d₂, which is greater than the first distance d₁, when the loading member 161 is in the actuated position.

In this embodiment the engagement elements 175a, b on the loading member 161 each comprise a recess, here in the form of a slot, in which the lower end of a respective one of the actuating members 163a, b is located, as will be described in more detail hereinbelow.

The actuating members 163a, b each comprise a flexible, elongate element, which has a length/which is greater both than the first distance d₁ and the second distance d₂ between the engagement elements 153a, b on the housing 111 and the engagement elements 175a, b on the loading member 161, the respective ends of which engage respective ones of the engagement elements 153a, b on the housing 111 and the engagement elements 175a, b on the loading member 161. With this configuration, as illustrated in FIG. 6, the actuating members 163a, b are each flexed outwardly when the actuating mechanism 109 is in the rest configuration, thus presenting the actuating members 163a, b for depression by operation of the grip members 115a, b by the user, and, when depressed, the actuating members 163a, b are straightened, such as to extend a greater distance in the axial direction, and, when this extension reaches the second distance d₂ between the engagement elements 153a, b on the housing 111 and the engagement elements 175a, b on the loading member 161, the canister 105 is actuated.

In this embodiment the actuating members 163a, b comprise resilient elements, here formed of a plastic, which adopt a flexed configuration, as illustrated in FIG. 6, when the actuating mechanism 109 is in the rest configuration.

Preferably, the components of the actuator are made from plastics materials.

In one embodiment the loading member 161 and the actuating members 163 could be formed as a single integral component, typically moulded from a plastics material, in which embodiment the engagement elements 175a, b are omitted.

Operation of the actuator will now be described hereinbelow.

The user first takes the actuator, as illustrated in FIG. 5, in one hand.

The user then takes the mouthpiece 113 in his/her lips, and, in co-ordination with an inhalation breath, actuates the inhaler by depressing the grip members 115a, b, which act to depress the actuating members 163a, b of the actuating mechanism 109.

As illustrated in FIG. 7, depression of the actuating members 163a, b causes the actuating members 163a, b to be straightened and thereby extended in the axial direction.

This straightening and extension in the axial direction of the actuating members 163a, b causes the loading member 161, and hence the body 123 of the canister 105 which is engaged thereby, to be driven downwardly in relation to the stationary valve stem 125 of the canister 105.

When this extension reaches the second distance d₂ between the engagement elements 153a, b on the housing 111 and the engagement elements 175a, b on the loading member 161, the canister 105 is actuated to deliver a pressurised medicament spray into and through the mouthpiece 113.

Following actuation, the inhaler is removed from the mouth, and the grip members 115a, b are released. On releasing the grip members 115a, b, the actuating members 163a, b, and hence the actuating mechanism 109, is returned by the valve return spring to the rest configuration, as illustrated in FIG. 6, ready for subsequent actuation.

It will be appreciated that the actuating mechanisms 9, 109 in the illustrated embodiments of the invention provide a mechanical advantage. That is to say, the manual force required to be applied by the user to operate the inhaler (by overcoming the return force of the valve return spring) is less than would otherwise be the case, such as in operation of a standard PMDI where the user has to push down on the base of the canister 5, 105 against the return force of the valve return spring.

As will be further appreciated, the loading member 61, 161 used in each illustrated embodiment could be replaced with a loading member corresponding to that used in the other embodiment. Moreover, the embodiment of FIGS. 1 to 4 could incorporate the grip members 115a, b of the embodiment of FIGS. 5 to 7.

In a yet further alternative embodiment, not shown, the loading member 61, 161 could be formed by a surface feature of the canister 5, 105.

Finally, it will be understood that the present invention has been described in its exemplary embodiments and can be modified in many different ways without departing from the scope of the invention as defined by the appended claims.

Also, as regards the provision of reference signs in the appended claims, it is to be understood that reference signs are provided only for illustrative purposes and are not intended to confer any limitation to the claimed invention.

What is claimed is:

1. An inhaler for delivering medicament by inhalation, comprising:
a canister which comprises a body which defines a chamber containing medicament and a valve stem which extends from the body and from which medicament is in use delivered on actuation of the canister; and
an actuator comprising a main body comprising a housing receiving the canister, and an actuating mechanism for actuating the canister;
wherein the actuating mechanism comprises a loading member which engages or is comprised in the canister, and at least one actuating member which is depressible by a user to drive the loading member in an actuating direction from a first, rest position to a second, actuated position in which the canister is actuated to deliver medicament;
wherein the at least one actuating member is a flexible element which has a first, flexed configuration when the loading member is in the first, rest position and, on depression, is extended in the actuating direction, such as to drive the loading member in the actuating direction from the first, rest position to the second, actuated position.

2. The inhaler of claim 1, wherein the main body includes at least one engagement element which engages the at least one actuating member.

3. The inhaler of claim 2, wherein the housing of the main body includes the at least one engagement element.

4. The inhaler of claim 2, wherein the least one engagement element on the main body engages an end of the at least one actuating member.

5. The inhaler of claim 1, wherein the loading member includes at least one engagement element which engages the at least one actuating member.

6. The inhaler of claim 5, wherein the at least one engagement element on the loading member engages an end of the at least one actuating member.

7. The inhaler of claim 1, wherein the body of the canister includes a base and a head, and the loading member is attached to the head of the body of the canister.

8. The inhaler of claim 1, wherein the loading member includes at least one engagement element which engages the at least one actuating member, wherein the body of the canister includes a base and a head, and the loading member is attached to the head of the body of the canister, wherein the loading member comprises a sleeve in which the head of the body of the canister is fixed and a body section which includes the at least one engagement element.

9. The inhaler of claim 1, wherein the body of the canister includes a base and a head, and the loading member is located over the base of the body of the canister.

10. The inhaler of claim 1, wherein the loading member includes at least one engagement element which engages the at least one actuating member, wherein the body of the canister includes a base and a head, and the loading member is located over the base of the body of the canister, wherein the loading member comprises a sleeve which fits about an outer peripheral surface of the body of the canister and includes the at least one engagement element, and an end section at one end of the sleeve which engages the base of the body of the canister.

11. The inhaler of claim 10, wherein the at least one engagement element is disposed at the other end of the sleeve of the loading member.

12. The inhaler of claim 10, wherein the sleeve of the loading member extends substantially to the head of the body of the canister.

13. The inhaler of claim 1, wherein the at least one actuating member is an elongate element.

14. The inhaler of claim 13, wherein the at least one actuating member is a resilient element which adopts the flexed configuration when not depressed.

15. The inhaler of claim 1, wherein the housing includes at least one lateral opening in which the at least one actuating member is disposed for depression by the user.

16. The inhaler of claim 1, wherein the main body further comprises at least one grip member which is movably disposed relative to the housing, such as, on depression, to engage the at least one actuating member in actuating the actuating mechanism.

17. The inhaler of claim 16, wherein the at least one grip member is pivotally coupled to the housing.

18. The inhaler of claim 1, wherein the actuating mechanism comprises first and second actuating members.

19. The inhaler of claim 18, wherein the actuating members are disposed to opposite lateral sides of the housing.

20. The inhaler of claim 1, wherein the main body includes a nozzle block which receives the valve stem of the canister.

21. The inhaler of claim 1, wherein the housing includes an outlet member through which the user in use inhales.

22. The inhaler of claim 21, wherein the outlet member is a mouthpiece.

23-25. (canceled)
